(19) United States
(12) Patent Application Publication
(10) Pub. No.: US 2025/0042834 A1
(43) Pub. Date: Feb. 6, 2025

(54) Fischer Esterification of Lignin-Derived Material

(71) Applicant: SixRing Inc., Calgary (CA)

(72) Inventors: Matthew DEWIT, Calgary (CA); Andrew C. CORBETT, Calgary (CA); Markus WEISSENBERGER, Calgary (CA)

(21) Appl. No.: 18/793,609

(22) Filed: Aug. 2, 2024

(30) Foreign Application Priority Data
Aug. 4, 2023 (CA) 3208558

(51) Int. Cl. C07C 67/08

(52) U.S. Cl. CPC C07C 67/08

(57) ABSTRACT

A method for converting a lignin-derived material into at least one esterified lignin derivative, said method comprising the steps of:
providing said lignin-derived material selected from the group consisting of: lignin monomers; lignin depolymerization products; and combinations thereof;
providing an acidic composition having a pH of less than 1, said acidic composition comprising:
   an acid selected from the group consisting of: sulfuric acid; an alkylsulfonic acid; and an arylsulfonic acid; and
   an alcohol selected from the group consisting of C₁-C₈ linear alcohol and C₃-C₈ branched alcohol and mixtures thereof;
combining said lignin-derived material with said acidic composition into a reaction mixture;
heating up said mixture to a temperature ranging from 25° C. to 120° C.; and
allowing sufficient time of reaction to convert at least a portion of said lignin-derived material into said at least one esterified lignin derivative.

### FIELD OF THE INVENTION

The present invention is directed to a method of converting lignin from biomass into valuable smaller chemicals, more specifically, in one instance, there is provided a method to convert lignin obtained using a modified Caro's acid delignification process into small valuable chemicals.

### BACKGROUND OF THE INVENTION

Petroleum is the cornerstone of the present chemical industry. Not only is it the most commonly used fuel in transportation, heating oils and electricity generation but it is also the primary raw material for the overwhelming majority of the basic chemicals used in plastics, adhesives and whole variety of synthetic materials just to name a few. The ever-growing demands and limits of the availability of this non-renewable resource is forcing the chemical industry to increase their research on the use of renewable resources as an alternative to petroleum.

Lignocellulosic biomass such as, but not limited to wood, grasses and other plant materials, contains three main components: cellulose fibers; lignin; and hemicelluloses. Pulping of lignocellulosic biomass has a primary goal to separate the fibers from the lignin. Lignin is a three-dimensional polymer which figuratively acts as a mortar to hold all the fibers together within the plant. Its presence in finished pulp is undesirable and adds nothing to the finished product.

Lignin accounts for, in some biomass, up to 30 percent of the lignocellulose biomass, and has a great potential to replace at least a portion of the petroleum-based chemical products. However, it is still greatly underused as such an alternative. Lignin is the second most abundant organic natural material encountered in nature. However, approximately 98% of it is still simply burned to provide heat or used in the production of energy.

Lignin is made up of various aromatic compounds and its complexity comes from the diversity and degree of crosslinking between the various monomeric units which comprises it. These are called lignolsand fall under one of three main categories: coniferyl alcohol; sinapyl alcohol; and paracoumaryl alcohol.

Aromatic compounds are normally extracted from petroleum and can be used in the production of a variety of high value products including but not limited to adhesives, drugs and paints. Consequently, the potential value locked up in lignin and its various monomeric constituents is quite high as it is the only naturally occurring source of such a large number of aromatic compounds.

The depolymerization of lignin into its various constituent monomeric building blocks is a major focus of several research groups as the subsequent uses of those monomers can open the doorway to a multitude of plant-derived chemical products. The yields of the lignin-originating aromatic monomers are largely dependent on the delignification method employed as well as the biomass used. Not all biomasses contain the same lignin content. Further, since lignin is a highly complex biopolymer, its origination from different biomasses means that the ratio of the lignin-originating aromatic monomers will vary from plant to plant.

The isolation of the individual lignin-originating aromatic monomers from complex mixtures following delignification of biomass is still a substantial challenge to the industry. Lignin condensation is a particular challenge which hinders the isolation of lignin constituents. The current lignin depolymerization techniques include alkaline oxidation, fast pyrolysis (used to maximize the liquid bio-oil product yield), hydrogenolysis, and hydrolysis.

European patent application EP2025735A1 teaches a one-step conversion of solid lignin to liquid products. More specifically, a method of converting a lignin material into a liquid product by treatment in a reaction medium comprising at least one C₁-C₂ carboxylic acid and the liquid product obtainable by the method.

U.S. Pat. No. 9,663,835B2 discloses a process and system for the efficient fractionation of lignocellulosic biomass into cellulose, hemicellulose sugars, lignin, and acetic acid. It states that the cellulose thus obtained is highly amorphous and can be readily converted into glucose using known methods. Fermentable hemicellulose sugars, low-molecular-weight lignin, and purified acetic acid are also major products of the process and system.

United States patent application US 2010/0121110A1 discloses a method for the breakdown of lignin which teaches a method for the direct production of molecules with a minimum molecular weight of 78 g/mol by the breakdown of lignin, lignin derivatives, lignin fragments, and/or lignin-containing substances or mixtures in the presence of at least one polyoxometallate and preferably in the presence of a radical scavenger in a liquid medium.

Japanese patent application JP2015089884A teaches a method for producing lignin monomers with high yield by decomposing a plant material containing a lignin component such as wood using a reaction agent which is easily available and has no problem with handleability. The method for producing lignin monomers uses a step of irradiating a mixture with microwave of a plant material containing a lignin component and a metal compound to decompose the plant material.

United States patent application US 2013/0232853A1 discloses a method of production of biobased chemicals, biofuels, and lignin residues from lignin sources, including waste lignin. This method may allow for selectively producing biobased chemicals, biofuels, and lignin residues from lignin sources using certain processing methods. The methods for production of these biobased chemicals, biofuels, and lignin residues may be provided by chemical-induced processing, catalytic oxidative lignin depolymerization processing, and catalytic hydroprocessing. Further, the catalytic hydroprocessing from processes including catalytic reduction processing, catalytic hydrodeoxygenation processing, and/or catalytic/dehydrogenation processing may also be used. The method described herein also provides a means in which waste from the process(es) may be reduced and/or recycled.

United States patent application US2016/0130202A1 discloses methods for the production and isolation of a monomer from a biopolymer. The method includes extracting a biopolymer from a biopolymer source and depolymerizing the biopolymer into a monomer. Also disclosed are methods for the production and isolation of a monomer from corn lignin.

Because of the heterogeneity of lignin and the substantial issues caused by the re-condensation of lignin monomers, there has not been a satisfactory approach to extract lignin from biomass and to further convert the extracted lignin to value added chemicals. Alkali lignin is particularly susceptible to condensation reaction. Since alkaline pulping represents the most widespread delignification and pulping processes across the world, the majority of the lignin thus extracted is not salvageable for further chemical processes and is typically used as a source of heat as it is simply burned.

One of the drawbacks of the lignin obtained through a kraft delignification or through the sulfite process is largely still polymerized and thus will not be useful in generating small molecules. Pyrolysis, on the other hand, is a method to produce lignin-derived molecules from lignocellulosic biomass. Conventional pyrolysis oil generates aldehydes which can polymerize over time and thus render such bio-oil unstable over time. Most bio-oils generated from pyrolysis have the same drawbacks. Their delignification process yields bio-oil which contains aldehydes, their aldehyde content makes them unstable for long-term storage. Pyrolysis oil also has other drawbacks which include: having a high oxygen content (making them less desirable for combustion in engines); they are largely non-volatile; and they may be corrosive.

In light of the state of the art, there still exists a need for lignin valorization, more specifically for a method capable of converting lignin depolymerization products into higher value chemicals.

### SUMMARY OF THE INVENTION

The inventors have surprisingly discovered that a mixture of valuable aromatic and aliphatic esters could be produced from lignin-originating aromatic monomers obtained from the delignification of biomass performed using a modified Caro's acid (i.e. H₂SO₄, in the presence of a modifier and a source of peroxide).

LHDO obtained from delignification of lignocellulosic biomass material using a modified Caro's acid, overcomes the problem caused by the presence of aldehyde by circumventing the production thereof. The oxidizing power of modified Caro's acid used favors the production of carboxylic acids and allows to achieve complete or very near to complete oxidation of the LHDO. Upon analysis, the aldehyde levels are below detection limits. According to a preferred embodiment of the present invention, the LHDO comprises lignin-derived material selected from the group consisting of: lignin monomers (20 to 50 wt. % of said lignin-derived material); lignin depolymerization products (50 to 80 wt. % of said lignin-derived material), wherein lignin depolymerization product is not a monomer but a soluble lignin-derivative, i.e. a breakdown compound.

However, a difficulty arose when wanting to extract the lignin depolymerization products present in the liquid recovered from a modified Caro's acid-driven delignification of biomass material. The various lignin monomers obtained from such a process were found to be hydrophilic and thus miscible with the remaining sulfuric acid present in the liquid recovered.

According to an aspect of the present invention, the inventors have developed a method which overcomes both the difficulties caused by the presence of a strong acid, inorganic impurities (such as sulfate salts, chlorides) and water in the liquid recovered but can also allow for the synthesis of various diester compounds and facilitate the recovery of such from a stream containing lignin depolymerization compounds as well as dissolved hemicellulose. It was surprisingly and unexpectedly discovered that a mixture of valuable aromatic and aliphatic esters could be produced from lignin-originating aromatic monomers obtained from the delignification of biomass performed using a modified Caro's acid (i.e. H₂SO₄, in the presence of a modifier and a source of peroxide).

According to one aspect of the present invention, there is provided a method to convert lignin-derived material (i.e. lignin depolymerization products comprising: lignin oligomers; and lignin monomers) into smaller molecules which are considered more valuable. According to a preferred embodiment of the present invention, the lignin-derived material obtained through the delignification of lignocellulosic feedstock (or biomass) by the methods and process disclosed herein include but are not limited to: lignin monomers; lignin depolymerization products such as: vanillic acid; malonic acid; maleic acid; succinic acid; oxalic acid; and 4-hydroxybenzoic acid. Preferably, the lignin-derived material forms part of the solubilized lignin and hemicellulose depolymerized organics (LHDO) stream resulting from a delignification of a lignocellulosic biomass through the use of a modified Caro's acid. Preferably, said lignin-hemicellulose depolymerized organics (LHDO) is a composition comprising: a strong acid and said lignin-derived material; said lignin-derived material comprises: lignin monomers (20 to 50 wt. %); lignin depolymerization products (50 to 80 wt. %).

The inventors have discovered that attempts to extract some of the lignin-derived material prior to esterification led to very poor yields. The one step that is deemed of some use was to concentrate the LHDO by reducing the water content which in turn would increase the efficiency of the esterification reaction.

According to one aspect of the present invention, there is provided a method to convert lignin and/or lignin fragments and/or lignin depolymerization products into smaller molecules which are considered more valuable. According to a preferred embodiment of the present invention, the lignin depolymerization products (also referred to as lignin-derived material) obtained through the delignification of lignocellulosic feedstock (or biomass) by the methods and process disclosed herein include but are not limited to: lignin monomers; lignin depolymerization products such as: vanillic acid; syringic acid; and 4-hydroxybenzoic acid.

According to an aspect of the present invention there is provided a method for converting a lignin-derived material into at least one esterified lignin derivative, said method comprising the steps of:
providing said lignin-derived material selected from the group consisting of: lignin monomers; lignin depolymerization products; and combinations thereof;
providing an acidic composition having a pH of less than 1, said acidic composition comprising:
   an acid selected from the group consisting of: sulfuric acid; an alkylsulfonic acid; and an arylsulfonic acid; and
   an alcohol selected from the group consisting of C₁-C₈ linear alcohol and C₃-C₈ branched alcohol and mixtures thereof;
combining said lignin-derived material with said acidic composition into a reaction mixture;
heating up said mixture to a temperature ranging from 25° C. to 120° C.; and
allowing sufficient time of reaction to convert at least a portion of said lignin-derived material into said at least one esterified lignin derivative.

Preferably, the alcohol is selected from the group consisting of: methanol; ethanol; n-propanol; isopropanol; n-butanol; isobutanol; n-pentanol; neo-pentanol; isopentanol; isoamyl alcohol and mixtures thereof. Preferably, the alcohol is n-butanol.

Preferably, the alcohol and the sulfuric acid are present in a molar ratio ranging from 2.85:1 (alcohol:sulfuric acid) to 10:1 (alcohol:sulfuric acid). More preferably, the alcohol and the sulfuric acid are present in a molar ratio ranging from 3:1 (alcohol:sulfuric acid) to 5:1 (alcohol:sulfuric acid).

According to a preferred embodiment of the present invention, the lignin-containing material results from a delignification reaction of a lignocellulosic material using a modified Caro's acid.

According to a preferred embodiment of the present invention, the at least one esterified lignin derivative is selected from the group consisting of: alkyl malonate; alkyl maleate; alkyl succinate; alkyl oxalate; dialkyl malonate; dialkyl maleate; dialkyl succinate; dialkyl oxalate; alkyl vanillate and alkylparaben. Preferably, said at least one esterified lignin derivative is selected from the group consisting of: dibutyl malonate; dibutyl maleate; dibutyl succinate; and dibutyl paraben.

According to a preferred embodiment of the present invention, the alkylsulfonic acid is selected from the group consisting of: methanesulfonic acid; ethanesulfonic acid; propanesulfonic acid and combinations thereof.

According to a preferred embodiment of the present invention, the arylsulfonic acid is selected from the group consisting of: toluenesulfonic acid; benzenesulfonic acid; and combinations thereof.

According to another aspect of the present invention there is provided a method to convert LHDO into at least one esterified lignin derivative and to recover such, said method comprising the steps of:
providing a LHDO comprising a lignin-containing material resulting from a delignification reaction of a lignocellulosic material using a modified Caro's acid, wherein said lignin-containing material comprises a total acid content ranging from 35-70%, preferably, said lignin-containing material is present in an amount of at least 3%;
   wherein said lignin-derived material comprises: lignin monomers (20 to 50 wt. %); lignin depolymerization products (50 to 80 wt. %);
optionally, removing at least a portion of water present in said LHDO;
adding the LHDO to a flask containing magnetic stir bar;
concentrating the material using a rotary evaporator until all volatile solvent was removed;
weighing the residue, and
adding a required mass of alcohol solvent (preferably 1:1 or 8:1 alcohol:LHDO by weight);
placing the mixture in an oil bath on a heating stir plate, where the bath has a temperature set to either 25 or 100° C.;
stirring the reaction mixture at the desired temperature for up to 16 hours;
optionally, cooling the reaction mixture;
filtering the reaction mixture to remove precipitated solids;
rinsing the filtered precipitated solids with additional alcohol, collecting, drying and weighing said solids;
concentrating the filtrate by evaporation and then transferring a resulting concentrated filtrate to a separatory funnel;
adding water and ethyl acetate, and extracting the product into an organic phase;
optionally repeating the extracting step;
collecting an organic phase;
extracting an aqueous phase;
optionally, combining the organic phases and transferring the combined organic phases back into the separatory funnel,
optionally, washing the combined organic phases with two portions of a pH 2 sulfate buffer solution;
optionally, drying the organic phase over MgSO₄, filtered into a round bottom flask, and evaporating the remaining volatiles; and
optionally, weighing the residue and calculating a yield.

Preferably, the LHDO has an acid content ranging from 40-45% prior to the addition into the flask.

According to a preferred embodiment of the present invention, said lignin monomers are present in an amount ranging from 20 to 50 wt. % of said lignin-derived material

According to a preferred embodiment of the present invention, said lignin depolymerization products are present in an amount ranging from 50 to 80 wt. % of said lignin-derived material.

According to a preferred embodiment of the present invention, there is provided a method to generate various aliphatic ester compounds and/or aromatic ester compounds by reacting lignin with a composition of sulfuric acid and an alcohol.

According to a preferred embodiment of the present invention, there is provided a method to generate various aliphatic ester compounds selected from the group consisting of: dialkyl malonate; dialkyl maleate; dialkyl succinate; and dialkyl oxalate.

According to a preferred embodiment of the present invention, there is provided a method to generate various aromatic ester compounds selected from the group consisting of: alkyl vanillate and alkylparaben.

According to a preferred embodiment of the present invention, the feedstock which can be employed in the process include but is not limited to: raw & concentrated liquid Lignin-Hemicellulose-Depolymerization-Organics (LHDO); kraft lignin; alkali lignin; and the like.

### DETAILED DESCRIPTION OF THE PRESENT INVENTION

According to a preferred embodiment of the present invention, a lignocellulosic biomass feedstock is delignified using a modified Caro's acid. The resulting delignification yields a stream of cellulose and a stream of solubilized lignin and hemicellulose depolymerized organics (LHDO). Preferably, said lignin-hemicellulose depolymerized organics (LHDO) is a composition comprising: a strong acid and said lignin-derived material; said lignin-derived material comprises: lignin monomers (20 to 50 wt. %); lignin depolymerization products (50 to 80 wt. %). The terms lignin depolymerization products or material may be used interchangeably herein with the term lignin oligomers, in either instance they are meant to distinguish lignin-derived material which are not considered to be lignin monomers.

According to a preferred embodiment of the present invention, the LHDO obtained from a delignification reaction of lignocellulosic biomass using a modified Caro's acid, comprises what can be considered as a bi-modal lignin-derived product distribution. There is a large concentration of compounds in the C₃-C₁₀ range and another large concentration of compounds in the C₁₂-C₃₀ range. Preferably, esterification reactions (especially with alcohols as large as butanol) are meant to increase the molecular weight of lighter lignin-derived material (such as lignin monomers) as well as react with the carboxylic acid groups and thus make them larger and less hydrophilic. Also esterification allows to obtain and isolate various esters of lignin monomers.

Preferably, to achieve such streams, the biomass comprising lignin, hemicellulose and cellulose fibers may be mechanically treated to reduce particle size prior to contacting it to a modified Caro's acid.

Preferably, to achieve such streams, the biomass comprising lignin, hemicellulose and cellulose fibers is exposed to a modified Caro's acid composition having a pH of less than 1, selected from the group consisting of: composition A; composition B; composition C; composition D; composition E; composition F; composition G; composition H; composition I; and composition J; wherein said composition A comprises:
sulfuric acid;
a compound comprising an amine moiety and a sulfonic acid moiety; and
a peroxide; and wherein sulfuric acid, said compound comprising an amine moiety and a sulfonic acid moiety and said peroxide are present in a molar ratio of no less than 1:1:1;
   wherein said composition B comprises:
sulfuric acid;
a compound comprising an amine moiety;
a compound comprising a sulfonic acid moiety; and
a peroxide; wherein sulfuric acid and said a compound comprising an amine moiety and said compound comprising a sulfonic acid moiety are present in a molar ratio of no less than 1:1:1;
   wherein said composition C comprises:
an alkylsulfonic acid; and
a peroxide; wherein said alkylsulfonic acid and said peroxide are present in a molar ratio of no less than 1:1;
   wherein said composition D comprises:
sulfuric acid;
a heterocyclic compound; and
a peroxide; and wherein sulfuric acid and said a heterocyclic compound; are present in a molar ratio of no less than 1:1;
   wherein said composition E comprises:
sulfuric acid;
a modifying agent comprising a compound containing an amine group; and
a peroxide; and wherein sulfuric acid and said compound containing an amine group; are present in a molar ratio of no less than 1:1;
   wherein said composition F comprises:
sulfuric acid;
a modifying agent comprising an alkanesulfonic acid and
a peroxide; and wherein sulfuric acid and said alkanesulfonic acid are present in a molar ratio of no less than 1:1;
   wherein said composition G comprises:
sulfuric acid;
a substituted aromatic compound; and
a peroxide; and wherein sulfuric acid and said substituted aromatic compound; are present in a molar ratio of no less than 1:1;
   wherein said composition H comprises:
sulfuric acid;
a modifying agent comprising an arylsulfonic acid;
a peroxide; and
optionally, a compound containing an amine group; wherein sulfuric acid and said a arylsulfonic acid; are present in a molar ratio of no less than 1:1;
   wherein said composition I comprises:
sulfuric acid;
a heterocyclic compound;
an alkanesulfonic acid and
a peroxide; and wherein sulfuric acid and said a heterocyclic compound; are present in a molar ratio of no less than 1:1;
   wherein said composition J comprises:
sulfuric acid;
a carbonyl-containing nitrogenous base compound; and
a peroxide; and wherein sulfuric acid and said a carbonyl-containing nitrogenous base compound; are present in a molar ratio of no less than 1:1;
   for a period of time sufficient to remove substantially all of the lignin present on said biomass material. The process can be carried out for a varying duration of time depending on the particle size of the biomass being fed into the process. The process can last from 2 to 20 hours depending on that characteristic. Moreover, the temperature of the resulting mixture also has an impact on the duration of the process as the reaction is highly exothermic, precautions are taken to prevent a runaway degradation of the cellulose. This would result in a carbon black resulting product with no value. The process is preferably run at temperatures below 50° C., more preferably at temperatures below 40° C. The process of delignification is preferably performed with a cooling means adapted to control the heat generated by the chemical reaction of delignification and maintain the temperature to avoid an undesirable ‘runaway’ reaction.

Preferably, said sulfuric acid, said compound comprising an amine moiety and a sulfonic acid moiety and said peroxide are present in a molar ratio of no more than 15:1:1. Preferably, for a modified Caro's acid comprising sulfuric acid, peroxide and taurine (as the modifier component), the molar composition is as follows: H₂O:H₂O₂:H₂SO₄:Taurine in a molar ratio of 56:10:10:1. Preferably, for a modified Caro's acid comprising TEOA/MSA, the molar composition is as follows: H₂O:H₂O₂:H₂SO₄:TEOA:MSA in a molar ratio of 56:10:10:1:1.

According to a preferred embodiment of the approach to obtain low lignin cellulose, said sulfuric acid and said compound comprising an amine moiety and a sulfonic acid moiety are present in a molar ratio of no less than 3:1.

Preferably, said compound comprising an amine moiety and a sulfonic acid moiety is selected from the group consisting of: taurine; taurine derivatives; and taurine-related compounds.

According to a preferred embodiment of the approach to obtain low lignin cellulose, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl.

Preferably, said linear alkylaminosulfonic acid is selected form the group consisting of: methyl; ethyl (taurine); propyl; and butyl.

Preferably, branched aminoalkylsulfonic acid is selected from the group consisting of: isopropyl; isobutyl; and isopentyl.

According to a preferred embodiment of the approach to obtain low lignin cellulose, said compound comprising an amine moiety and a sulfonic acid moiety is taurine.

According to a preferred embodiment of the approach to obtain low lignin cellulose, said sulfuric acid and compound comprising an amine moiety and a sulfonic acid moiety are present in a molar ratio of no less than 3:1.

According to a preferred embodiment of the approach to obtain low lignin cellulose, said compound comprising an amine moiety is an alkanolamine is selected from the group consisting of: monoethanolamine; diethanolamine; triethanolamine; and combinations thereof.

According to a preferred embodiment of the approach to obtain low lignin cellulose, said compound comprising a sulfonic acid moiety is selected from the group consisting of: alkylsulfonic acids; arylsulfonic acids; and combinations thereof.

Preferably, said alkylsulfonic acid is selected from the group consisting of: alkylsulfonic acids where the alkyl groups range from C₁-C₆ and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Preferably, said arylsulfonic acid is selected from the group consisting of: toluenesulfonic acid; benzesulfonic acid; and combinations thereof.

According to a preferred embodiment of the approach to obtain low lignin cellulose, said alkylsulfonic acid; and said peroxide are present in a molar ratio of no less than 1:1.

Preferably, said compound comprising a sulfonic acid moiety is methanesulfonic acid.

According to a preferred embodiment of the approach to obtain low lignin cellulose (i.e. MCA cellulose), said Composition C may further comprise a compound comprising an amine moiety. Preferably, the compound comprising an amine moiety has a molecular weight below 300 g/mol. Preferably also, the compound comprising an amine moiety is a primary amine. More preferably, the compound comprising an amine moiety is an alkanolamine. Preferably, the compound comprising an amine moiety is a tertiary amine. According to a preferred embodiment of the approach to obtain low lignin cellulose, the alkanolamine is selected from the group consisting of: monoethanolamine; diethanolamine; triethanolamine; and combinations thereof. Preferably, the alkanolamine is triethanolamine.

According to a preferred embodiment of the approach to obtain low lignin cellulose, said in Composition C, said sulfuric acid and said a compound comprising an amine moiety and said compound comprising a sulfonic acid moiety are present in a molar ratio of no less than 1:1:1.

Preferably, in Composition C, said sulfuric acid, said compound comprising an amine moiety and said compound comprising a sulfonic acid moiety are present in a molar ratio ranging from 28:1:1 to 2:1:1.

Preferably, in Composition C, said compound comprising an amine moiety is triethanolamine and said compound comprising a sulfonic acid moiety is methanesulfonic acid.

According to preferred embodiment of the present invention, the modified Caro's acid (as disclosed in Canadian patent application 3,128,678) comprises: sulfuric acid; a heterocyclic compound and a peroxide; and wherein sulfuric acid and said a heterocyclic compound; are present in a molar ratio of no less than 1:1. Preferably, the sulfuric acid and said heterocyclic compound are present in a molar ratio ranging from 28:1 to 2:1 More preferably, the sulfuric acid and heterocyclic compound are present in a molar ratio ranging from 24:1 to 3:1. Preferably, the sulfuric acid and heterocyclic compound are present in a molar ratio ranging from 20:1 to 4:1. More preferably, the sulfuric acid and heterocyclic compound are present in a molar ratio ranging from 16:1 to 5:1. Preferably, the sulfuric acid and heterocyclic compound are present in a molar ratio ranging from 12:1 to 6:1. Also preferably, said heterocyclic compound has a molecular weight below 300 g/mol. Also preferably, said heterocyclic compound has a molecular weight below 150 g/mol. More preferably, said heterocyclic compound is a secondary amine. According to a preferred embodiment of the present invention, said heterocyclic compound is selected from the group consisting of: imidazole; triazole; and N-methylimidazole.

According to preferred embodiment of the present invention, the modified Caro's acid (as disclosed in Canadian patent application 3,128,677) comprises: sulfuric acid; a modifying agent comprising a compound containing an amine group and a peroxide; and wherein sulfuric acid and said compound containing an amine group; are present in a molar ratio of no less than 1:1. Preferably, the sulfuric acid and said compound containing an amine group are present in a molar ratio ranging from 28:1 to 2:1. More preferably, the sulfuric acid and compound containing an amine group are present in a molar ratio ranging from 24:1 to 3:1. Preferably, the sulfuric acid and compound containing an amine group are present in a molar ratio ranging from 20:1 to 4:1. More preferably, the sulfuric acid and compound containing an amine group are present in a molar ratio ranging from 16:1 to 5:1. Preferably, the sulfuric acid and compound containing an amine group are present in a molar ratio ranging from 12:1 to 6:1. According to a preferred embodiment of the present invention, the modifying agent is selected in the group consisting of: TEOA; MEOA; pyrrolidine; DEOA; ethylenediamine; diethylamine; triethylamine; morpholine; MEA-triazine; and combinations thereof. According to a more preferred embodiment of the present invention, the modifying agent is TEOA; MEOA; pyrrolidine; DEOA; ethylenediamine; triethylamine.

According to preferred embodiment of the present invention, the modified Caro's acid (as disclosed in Canadian patent application 3,128,676) comprises: sulfuric acid; a modifying agent comprising an alkanesulfonic acid and a peroxide; and wherein sulfuric acid and said alkanesulfonic acid are present in a molar ratio of no less than 1:1. Preferably, said alkanesulfonic acid is selected from the group consisting of: alkanesulfonic acids where the alkyl groups range from C₁-C₆ and are linear or branched; and combinations thereof. Preferably, said alkanesulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkanesulfonic acid is methanesulfonic acid. Also preferably, said alkanesulfonic acid has a molecular weight below 300 g/mol. Also preferably, said alkanesulfonic acid has a molecular weight below 150 g/mol. Preferably, the sulfuric acid and said alkanesulfonic acid and are present in a molar ratio ranging from 28:1 to 2:1. More preferably, the sulfuric acid and alkanesulfonic acid are present in a molar ratio ranging from 24:1 to 3:1. Preferably, the sulfuric acid and alkanesulfonic acid are present in a molar ratio ranging from 20:1 to 4:1. More preferably, the sulfuric acid and alkanesulfonic acid are present in a molar ratio ranging from 16:1 to 5:1. According to a preferred embodiment of the present invention, the sulfuric acid and alkanesulfonic acid are present in a molar ratio ranging from 12:1 to 6:1.

According to preferred embodiment of the present invention, the modified Caro's acid (as disclosed in Canadian patent application 3,128,675) comprises: sulfuric acid; a substituted aromatic compound and a peroxide; and wherein sulfuric acid and said substituted aromatic compound; are present in a molar ratio of no less than 1:1. Preferably, the substituted aromatic compound comprises at least two substituents. More preferably, at least one substituent is an amine group and at least one of the other substituent is a sulfonic acid moiety. According to a preferred embodiment, the substituted aromatic compound comprises three or more substituent. According to a preferred embodiment of the present invention, the substituted aromatic compound comprises at least a sulfonic acid moiety. According to another preferred embodiment of the present invention, the substituted aromatic compound comprises an aromatic compound having a sulfonamide substituent, where the compound can be selected from the group consisting of: benzenesulfonamides; toluenesulfonamides; substituted benzenesulfonamides; and substituted toluenesulfonamides. Preferably, the sulfuric acid and said substituted aromatic compound and are present in a molar ratio ranging from 28:1 to 2:1. More preferably, the sulfuric acid and substituted aromatic compound are present in a molar ratio ranging from 24:1 to 3:1. Preferably, the sulfuric acid and substituted aromatic compound are present in a molar ratio ranging from 20:1 to 4:1. More preferably, the sulfuric acid and substituted aromatic compound are present in a molar ratio ranging from 16:1 to 5:1. Preferably, the sulfuric acid and substituted aromatic compound are present in a molar ratio ranging from 12:1 to 6:1.

According to preferred embodiment of the present invention, the modified Caro's acid (as disclosed in Canadian patent application 3,128,674) comprises: sulfuric acid; a modifying agent comprising an arylsulfonic acid; a peroxide; and optionally, a compound containing an amine group; wherein sulfuric acid and said a arylsulfonic acid; are present in a molar ratio of no less than 1:1. Preferably, the compound containing an amine group is selected from the group consisting of: imidazole; N-methylimidazole; triazole; monoethanolamine (MEOA); diethanolamine (DEOA); triethanolamine (TEOA); pyrrolidine and combinations thereof. According to a preferred embodiment of the present invention, sulfuric acid and the peroxide are present in a molar ratio of approximately 1:1. Preferably, the sulfuric acid and said arylsulfonic acid and are present in a molar ratio ranging from 28:1 to 2:1. More preferably, the sulfuric acid and arylsulfonic acid are present in a molar ratio ranging from 24:1 to 3:1. Preferably, the sulfuric acid and arylsulfonic acid are present in a molar ratio ranging from 20:1 to 4:1. More preferably, the sulfuric acid and arylsulfonic acid are present in a molar ratio ranging from 16:1 to 5:1. According to a preferred embodiment of the present invention, the sulfuric acid and arylsulfonic acid are present in a molar ratio ranging from 12:1 to 6:1. Also preferably, said arylsulfonic acid has a molecular weight below 300 g/mol. Also preferably, said arylsulfonic acid has a molecular weight below 150 g/mol. Even more preferably, said arylsulfonic acid is selected from the group consisting of: orthanilic acid; metanilic acid; sulfanilic acid; toluenesulfonic acid; benzenesulfonic acid; and combinations thereof.

According to preferred embodiment of the present invention, the modified Caro's acid (as disclosed in Canadian patent application 3,128,673) comprises: sulfuric acid; a heterocyclic compound; an alkanesulfonic acid and a peroxide; and wherein sulfuric acid and said a heterocyclic compound; are present in a molar ratio of no less than 1:1. Preferably, said aqueous acidic composition comprising: sulfuric acid; a heterocyclic compound; an arylsulfonic acid; and wherein sulfuric acid and said a heterocyclic compound; are present in a molar ratio of no less than 1:1. Preferably, the arylsulfonic acid is toluenesulfonic acid.

Preferably, the sulfuric acid, the heterocyclic compound and the alkanesulfonic acid are present in a molar ratio ranging from 28:1:1 to 2:1:1. More preferably, the sulfuric acid the heterocyclic compound and the alkanesulfonic acid are present in a molar ratio ranging from 24:1:1 to 3:1:1. Preferably, the sulfuric acid, the heterocyclic compound and the alkanesulfonic acid are present in a molar ratio ranging from 20:1:1 to 4:1:1. More preferably, the sulfuric acid, the heterocyclic compound and the alkanesulfonic acid are present in a molar ratio ranging from 16:1:1 to 5:1:1. According to a preferred embodiment of the present invention, the sulfuric acid and heterocyclic compound are present in a molar ratio ranging from 12:1:1 to 6:1:1. Also preferably, said heterocyclic compound has a molecular weight below 300 g/mol. Also preferably, said heterocyclic compound has a molecular weight below 150 g/mol. Even more preferably, said heterocyclic compound is selected from the group consisting of: imidazole; triazole; n-methylimidazole; and combinations thereof. Preferably, the alkanesulfonic acid is selected from the group consisting of: alkylsulfonic acids where the alkyl groups range from C₁-C₆ and are linear or branched; and combinations thereof. Preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof. More preferably, said alkylsulfonic acid is methanesulfonic acid.

According to preferred embodiment of the present invention, the modified Caro's acid (as disclosed in Canadian patent application 3,128,672) comprises: sulfuric acid; a carbonyl-containing nitrogenous base compound and a peroxide; and wherein sulfuric acid and said a carbonyl-containing nitrogenous base compound; are present in a molar ratio of no less than 1:1. According to a preferred embodiment of the present invention, the carbonyl-containing nitrogenous base compound is selected from the group consisting of: caffeine; lysine; creatine; glutamine; creatinine; 4-aminobenzoic acid; glycine; NMP (N-methyl-2-pyrrolidinone); histidine; DMA (N,N-dimethylacetamide); arginine; 2,3-pyridinedicarboxylic acid; hydantoin; and combinations thereof. Preferably, the sulfuric acid and said carbonyl-containing nitrogenous base compound and are present in a molar ratio ranging from 28:1 to 2:1. More preferably, the sulfuric acid and carbonyl-containing nitrogenous base compound are present in a molar ratio ranging from 24:1 to 3:1. Preferably, the sulfuric acid and carbonyl-containing nitrogenous base compound are present in a molar ratio ranging from 20:1 to 4:1. More preferably, the sulfuric acid and carbonyl-containing nitrogenous base compound are present in a molar ratio ranging from 16:1 to 5:1. According to a preferred embodiment of the present invention, the sulfuric acid and carbonyl-containing nitrogenous base compound are present in a molar ratio ranging from 12:1 to 6:1.

Preferably, exposing said biomass to said modified Caro's acid composition will allow the delignification reaction to occur and remove over 90 wt % of said lignin and hemicellulose from said biomass. Preferably, the delignification reaction is carried out at a temperature below 55° C. by a method selected from the group consisting of:
adding water into said vessel;
adding biomass into said vessel; and
using a heat exchanger.

Preferably, said sulfuric acid, said compound comprising an amine moiety and a sulfonic acid moiety and said peroxide are present in a molar ratio of no less than 1:1:1. Also preferably, said sulfuric acid, said compound comprising an amine moiety and a sulfonic acid moiety and said peroxide are present in a molar ratio of no more than 15:1:1. Preferably, said sulfuric acid and said compound comprising an amine moiety and a sulfonic acid moiety are present in a molar ratio of no less than 3:1.

Preferably, said modifier compound comprising an amine moiety and a sulfonic acid moiety is selected from the group consisting of: taurine; taurine derivatives; and taurine-related compounds. Preferably, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₃-C₅ branched alkyl. Preferably, said linear alkylaminosulfonic acid is selected form the group consisting of: methyl; ethyl (taurine); propyl; and butyl. Preferably, said branched aminoalkylsulfonic acid is selected from the group consisting of: isopropyl; isobutyl; and isopentyl.

Preferably, said sulfuric acid and compound comprising an amine moiety and a sulfonic acid moiety are present in a molar ratio of no less than 3:1.

Preferably, said compound comprising an amine moiety is an alkanolamine is selected from the group consisting of: monoethanolamine; diethanolamine; triethanolamine; and combinations thereof.

Preferably, said compound comprising a sulfonic acid moiety is selected from the group consisting of: alkylsulfonic acids and combinations thereof. More preferably, said alkylsulfonic acid is selected from the group consisting of: alkylsulfonic acids where the alkyl groups range from C₁-C₆ and are linear or branched; and combinations thereof. Yet even more preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Also preferably, said alkylsulfonic acid; and said peroxide are present in a molar ratio of no less than 1:1. Preferably, said compound comprising a sulfonic acid moiety is methanesulfonic acid.

Preferably, in Composition C, said sulfuric acid and said a compound comprising an amine moiety and said compound comprising a sulfonic acid moiety are present in a molar ratio of no less than 1:1:1. More preferably, in Composition C, said sulfuric acid, said compound comprising an amine moiety and said compound comprising a sulfonic acid moiety are present in a molar ratio ranging from 28:1:1 to 2:1:1.

According to a preferred embodiment of the present invention, the alcohol is selected from the group consisting of: C₁-C₈ linear alcohols and C₃-C₈ branched alcohols and mixtures thereof. Preferably, the alcohol is selected from the group consisting of: isoamyl alcohol, 2-butanol, isobutyl alcohol, 2-ethylhexanol, 2-octanol; methanol, ethanol, n-propanol, isopropanol, n-butanol, n-hexanol, n-octanol. More preferably, the alcohol is selected from the group consisting of: methanol, ethanol, n-propanol, isopropanol, n-butanol, n-hexanol, n-octanol and combinations thereof.

According to a preferred embodiment of the present invention, the ratio of alcohol:feedstock is present in a weight ratio ranges from 0.5:1 to 8:1. More preferably, the alcohol:feedstock weight ratio ranges from 1:1 to 6:1. Even more preferably, the alcohol:feedstock weight ratio ranges from 1:1 to 4:1. According to a preferred embodiment of the present invention, the ratio of alcohol:feedstock is present in a weight ratio of 1:1.

According to a preferred embodiment of the present invention, the duration of the reaction is up to 3 hours. According to another preferred embodiment of the present invention, the duration of the reaction is up to 6 hours. According to yet another preferred embodiment of the present invention, the duration of the reaction is up to 18 hours. According to yet another preferred embodiment of the present invention, the duration of the reaction is up to 24 hours.

According to a preferred embodiment of the present invention, the reaction is carried out at a temperature of 25° C. According to another preferred embodiment of the present invention, the reaction is carried out at a temperature of up to 40° C. According to another preferred embodiment of the present invention, the reaction is carried out at a temperature of up to 60° C. According to another preferred embodiment of the present invention, the reaction is carried out at a temperature of up to 80° C. According to another preferred embodiment of the present invention, the reaction is carried out at a temperature of up to 100° C. According to another preferred embodiment of the present invention, the reaction is carried out at a temperature of up to 120° C.

### EXAMPLES

### General Procedure for Esterification Reactions

LHDO feedstock obtained from a delignification of lignocellulosic biomass using a modified Caro's acid contains: dissolved lignin (present as: lignin monomers; lignin depolymerization products; and a combination thereof); dissolved hemicellulose; inorganic impurities (such as sulfate salts, chlorides); and water.

LHDO concentrate aims to reduce the presence of water and compared to the raw LHDO, it contains 30-40% less water. Of course, more water can be removed from the LHDO feedstock but as a cost to the overall process, the costs related water removal must be considered versus the benefit. Water removal has several benefits which include a more efficient esterification step as there is less water to impede the reaction.

The yield values are expressed in terms of percentage of dissolved lignin and hemicellulose that gets esterified and converted into the bio-oil. Higher yields means a greater percentage of the material as a whole gets converted into partially or fully esterified LHDO and becomes organic-soluble. Water content indicates how hydrophobic that material is. Even though the material is organic-soluble it can still be somewhat hydrophilic. Lower water content indicates a more hydrophobic product, while higher water content indicates less hydrophobic product. Preferably, lower water content is desirable.

Total Acid Number (TAN) gives an indication of the efficiency of the reaction. The definition of TAN is the mass in mg of KOH required to neutralize 1 g of oil, which means that the more acid the oil contains, the higher the TAN value will be. The starting LHDO contains a of lot of carboxylic acid compounds and would therefore have a very high TAN value (above 500). As these acids get converted into esters, they no longer react with KOH and so the TAN value will decrease, and so the more acid groups that get converted into esters, the lower the TAN value of the finished product will be. Lower TAN value means fewer carboxylic acids in the finished product which, in turn, means greater conversion efficiency. Yield values: above 60% is excellent, 50-60% is good, 30-50% is moderate, below 30% is poor. Water content values: above 1.5% is poor, between 0.5-1.5% is moderate, between 0.5-0.2% is good, below 0.2% is excellent. TAN values: above 150 is poor, between 100-150 is moderate, between 50-100 is good (matches pyrolysis oil), below 50 is excellent.

LHDO obtained from a delignification of lignocellulosic biomass using a modified Caro's acid as described hereinabove added to pre-weighed round bottom flask containing magnetic stir bar. Material was concentrated on a rotavap (bath temperature 50° C., vacuum gradually decreased to 1 mbar) until all volatile solvent was removed. Residue was weighed, and then the required mass of alcohol solvent was added (either 1:1 or 8:1 alcohol:LHDO by weight). The mixture was placed in an oil bath on a heating stir plate, and the bath temperature was set to either 25 or 100° C. (80° C. when methanol was used as the alcohol). For reactions carried out at 25° C., an air condenser was used, and for the reactions carried out at 80 or 100° C., a reflux condenser was used. In the LHDO, the acid is present in a concentration ranging from 30-70%, more preferably from 40-65%. More preferably, the raw LHDO has an acid content of between 40-45%. Based on these numbers, acid concentration of the reaction mixture will range from 3-35% depending on the dilution factor upon combining the LHDO with the alcohol.

The reaction mixture was then stirred at the desired temperature for 16 hours. After 16 hours, the reaction mixture was removed from the oil bath, left to cool, if necessary, and then filtered through a medium fritted filter to remove precipitated solids. The solids were rinsed with additional alcohol, collected, dried overnight in a 45° C. oven, and then weighed. The filtrate was concentrated on a rotary evaporator and then transferred to a separatory funnel. Water and ethyl acetate were added, and the product was extracted into the ethyl acetate phase. The organic phase was collected, and the aqueous phase was extracted two additional times with fresh ethyl acetate. The organic phases were combined and transferred back into the separatory funnel, where they were washed with two portions of a pH 2 sulfate buffer solution. The organic phase was then dried over MgSO4, filtered into a round bottom flask, and evaporated on a rotary evaporator to remove all volatiles. The residue was then weighed and the yield calculated.

TABLE 1
Yields of experiments carried out according to a preferred
embodiment of the present invention at 25° C.

| Alcohol | Ratio | Temperature | Yield | % H2O | TAN |
|---|---|---|---|---|---|
| MeOH | 1:1 | 25 | 26.07 | 1.42 | 97.9 |
| MeOH | 8:1 | 25 | 26.18 | 0.82 | 115.4 |
| EtOH | 1:1 | 25 | 38.74 | 1.23 | 95.2 |
| EtOH | 8:1 | 25 | 41.27 | 0.93 | 113.1 |
| iPrOH | 1:1 | 25 | 48.24 | 0.55 | 149.8 |
| iPrOH | 8:1 | 25 | 46.23 | 0.82 | 221.6 |
| nPrOH | 1:1 | 25 | 60.38 | 0.48 | 78.6 |
| nPrOH | 8:1 | 25 | 56.37 | 1.80 | 104.8 |
| nBuOH | 1:1 | 25 | 68.61 | 0.56 | 45.2 |
| nBuOH | 8:1 | 25 | 90.25 | 2.36 | 76.2 |

where iPrOH: isopropanol;
MeOH: methanol;
EtOH: ethanol:
nPrOH: n-propanol

The data summarized in table 1 indicates that upon exposure to a composition according to a preferred embodiment of the present invention, the lignin treated at 25° C. for 18 hours yielded over 2% diester compounds in every reaction. Notably, the longer chain alcohols provided the higher yields in diester compounds while the shorter chain alcohols yielded the lowest amounts. Also noteworthy of mention, the water content is very low, in most of the experiments the water content did not exceed 1%. This is important as the presence of water poisons the catalyst and contaminates the finished product. The finished product is the LHDO which comprises a mixture of fuel cuts. Low water content and low organic impurities (S and N) is desirable when converting LHDO into sustainable aviation fuel (SAF).

TABLE 2
Yields of experiments carried out according to a preferred
embodiment of the present invention at 100° C.

| Alcohol | Ratio | Temperature | Yield | % H2O | TAN |
|---|---|---|---|---|---|
| MeOH | 1:1 | 100 | 26.39 | 1.07 | 73.8 |
| MeOH | 8:1 | 100 | 27.87 | 1.03 | 76.7 |
| EtOH | 1:1 | 100 | 44.65 | 0.89 | 72.0 |
| EtOH | 8:1 | 100 | 39.06 | 0.78 | 56.1 |
| iPrOH | 1:1 | 100 | 49.51 | 0.83 | 133.5 |
| iPrOH | 8:1 | 100 | 55.95 | 0.75 | 96.0 |
| nPrOH | 1:1 | 100 | 60.59 | 1.13 | 60.4 |
| nPrOH | 8:1 | 100 | 58.80 | 1.03 | 52.0 |
| nBuOH | 1:1 | 100 | 101.12* | 0.75 | 85.6 |
| nBuOH | 8:1 | 100 | 110.31* | 2.21 | 127.0 |

*Residual solvent present

The data summarized in table 2 indicates that upon exposure to a composition according to a preferred embodiment of the present invention, the lignin treated at 100° C. for 18 hours yielded over 2% diester compounds in every reaction. Notably, the longer chain alcohols provided the higher yields in diester compounds while the shorter chain alcohols yielded the lowest amounts.

TABLE 3
Yields of experiments carried out according to a preferred embodiment
of the present invention using isopropanol as solvent and
using a feedstock concentrate for a duration of 18 hours

| | | | Esterified | Esterified | | | |
|---|---|---|---|---|---|---|---|
| | | Temp. | compounds | compounds | % | | |
| Alcohol | Ratio | (° C.) | Yield | Yield | H2O | TAN | Notes |
| iPrOH | 4:1 | 60 | 48.24 | 4.57 | 0.40 | 110.7 | |
| iPrOH | 8:1 | 60 | 48.45 | 4.59 | 0.38 | 109.9 | |
| iPrOH | 6:1 | 60 | 49.30 | 4.67 | 0.37 | 112.7 | |
| iPrOH | 6:1 | 77.5 | 48.56 | 4.60 | 0.39 | 101.0 | |
| iPrOH | 4:1 | 77.5 | 49.40 | 4.68 | 0.69 | 96.1 | |
| iPrOH | 3:1 | 60 | 52.67 | 4.99 | 0.41 | 115.7 | |
| iPrOH | 2:1 | 60 | 55.00 | 5.21 | 0.31 | 124.7 | |
| iPrOH | 2:1 | 60 | 38.00 | 4.63 | 0.52 | 144.0 | 5 wt % H2O in |
| | | | | | | | iPrOH |
| iPrOH | 3:1 | 60 | 48.87 | 4.40 | 0.48 | 135.5 | 5 wt % H2O in |
| | | | | | | | iPrOH |

The data summarized in table 3 indicates that upon exposure to a composition according to a number of preferred embodiments of the present invention, the feedstock concentrate (lignin depolymerization compounds) treated at various temperatures for 18 hours yielded over 4.5% diester compounds in every reaction.

TABLE 4
Yields of experiments carried out
according to a preferred embodiment
of the present invention using isopropanol
as solvent, using a feedstock
concentrate and comparing the yield
at different duration of reaction

| | | | | Esterified | | |
|---|---|---|---|---|---|---|
| | | | Temp. | compounds | | |
| Alcohol | Ratio | Time | (° C.) | Yield | % H2O | TAN |
| iPrOH | 2:1 | 24 h | 60 | 50.77 | 0.62 | 103.5 |
| iPrOH | 2:1 | 6 h | 60 | 50.03 | 0.52 | 120.4 |
| iPrOH | 2:1 | 18 h | 40 | 50.88 | 0.49 | 123.2 |

The data summarized in table 4 indicates that upon exposure to a composition according to a preferred embodiment of the present invention, the lignin treated at 60° C. for 6 to 24 hours yielded over 4.7% diester compounds in every reaction. The water content in each product is also quite low as it hovers around 0.5%.

TABLE 5
Yields of experiments carried out according to a preferred embodiment of the present
invention using various solvents for a duration of 18 hours and comparing the yield

| | | | Esterified | | | |
|---|---|---|---|---|---|---|
| | | Temp. | compounds | % | | |
| Alcohol | Ratio | (° C.) | Yield | H2O | TAN | Notes |
| EtOH | 6:1 | 60 | 38.00 | 0.61 | 71.9 | |
| iPrOH | 2:1 | 60 | 44.65 | 0.64 | 12.01 | recycled iPrOH used, contained 9.82 |
| | | | | | | wt % H2O |
| iPrOH | 3:1 | 60 | 52.26 | 0.77 | 13.65 | recycled iPrOH used, contained 9.82 |
| | | | | | | wt % H2O |
| iPrOH | 15:10 | 60 | 52.46 | 0.49 | 9.97 | |
| iPrOH | 1:1 | 60 | 53.94 | 0.56 | 10.73 | |
| iPrOH | 2:1 | 25 | 43.17 | 0.60 | 141.5 | |
| iPrOH | 3:1 | 25 | 42.43 | 0.82 | 171.6 | |
| nBuOH | 4:1 | 60 | 75.90 | 0.35 | 23.3 | product contains residual n- |
| | | | | | | Butanol that couldn't be removed |
| | | | | | | on rotavap |
| nBuOH | 2:1 | 60 | 81.38 | 1.35 | 55.3 | product contains residual n- |
| | | | | | | Butanol that couldn't be removed |
| | | | | | | on rotavap |

The data summarized in table 5 indicates that upon exposure to a composition according to a preferred embodiment of the present invention, the lignin treated at 100° C. for 18 hours yielded over 2% ester compounds in every reaction, and in some cases (n-butanol) the yields obtained were above 7%. Notably, the longer chain alcohols provided the higher yields in diester compounds while the shorter chain alcohols yielded the lowest amounts. The water content is also quite low as it is well below 1% in all but one case.

TABLE 6
Yields of experiments carried out according to a preferred embodiment
of the present invention using a raw feedstock and various solvents
for a duration of 18 hours and comparing the yield

| | | | Esterified | | |
|---|---|---|---|---|---|
| | | | compounds | | |
| Alcohol | Ratio | Temperature | Yield | % H2O | TAN |
| nPrOH | 4:1 | 60 | 36.84 | 0.95 | 97.9 |
| nPrOH | 2:1 | 60 | 32.20 | 0.39 | 71.7 |
| iPrOH | 2:1 | 60 | 25.65 | 0.73 | 203.8 |
| iPrOH | 3:1 | 60 | 24.91 | 0.61 | 176.5 |
| iPrOH | 4:1 | 60 | 24.38 | 1.18 | 176.5 |
| iPrOH | 6:1 | 60 | 25.12 | 0.59 | 171.5 |
| iPrOH | 15:10 | 60 | 24.91 | 0.67 | 226.8 |
| iPrOH | 1:1 | 60 | 20.58 | 1.12 | 303.8 |

The data summarized in table 6 indicates that upon exposure to a composition according to a preferred embodiment of the present invention, the lignin treated at 60° C. for 18 hours yielded over 2% diester compounds in almost every reaction.

TABLE 7
Yields of experiments carried out according to a preferred embodiment
of the present invention using a raw feedstock using isopropanol
for a duration of 18 hours and comparing the yield

| | | | Esterified | | |
|---|---|---|---|---|---|
| | | Temp | compounds | | |
| Alcohol | Ratio | (° C.) | Yield | % H2O | TAN |
| iPrOH | 2:1 | 40 | 22.27 | 1.70 | 261.4 |
| iPrOH | 3:1 | 40 | 23.64 | 1.40 | 210.4 |

The data summarized in table 7 indicates that upon exposure to a composition according to a preferred embodiment of the present invention, the lignin treated at 40° C. for 18 hours yielded over 2% diester compounds in every reaction.

TABLE 8
Yields of experiments carried out according to a preferred
embodiment of the present invention using a concentrated
LHDO feedstock using isobutanol for 18 hours at 60° C.

| | Esterified | | | |
|---|---|---|---|---|
| Ratio | compounds | | | |
| (alcohol:LHDO) | Yield | % H2O | TAN | Comments |
| 2:1 | 81.38 | 1.35 | 55.3 | product contains |
| | | | | residual n-Butanol |
| | | | | that couldn't be |
| | | | | removed on rotavap |
| 2:1 | 76.74 | 0.63 | 79.1 | |
| 4:1 | 70.51 | 0.61 | 49.3 | |

The data summarized in table 8 indicates that upon exposure to a composition according to a preferred embodiment of the present invention, the lignin-derived material from the LHDO concentrate treated at 60° C. for 18 hours yielded over 6% diester compounds in every reaction.

TABLE 9
Yields of experiments carried out according to a preferred embodiment of the present
invention using a concentrated LHDO feedstock using isobutanol for 18 hours at 55° C.

| | | | Esterified | | | |
|---|---|---|---|---|---|---|
| | Ratio | | compounds | % | | |
| alcohol | (alcohol:LHDO) | time | Yield | H2O | TAN | Comments |
| Methanol | 2:1 | 18 | 14.03 | 0.01 | 97.48 | |
| Methanol/ | 2:1 | 18 | 17.27 | 0.1 | 77.48 | |
| Methanol | | | | | | |
| Ethanol | 2:1 | 18 | 32.38 | 0.19 | 150.23 | |
| Ethanol/ | 2:1 | 18 | 31.84 | 0.06 | 102.03 | Double esterification; |
| Ethanol | | | | | | rotavap solvent as |
| | | | | | | much as possible, then |
| | | | | | | redissolve in fresh |
| | | | | | | EtOH and repeat |
| | | | | | | reaction, performing |
| | | | | | | full workup |
| Ethanol/ | 2:1 | 18 | 25.09 | 0.04 | 75.93 | Double esterification; |
| Ethanol | | | | | | rotavap solvent, |
| | | | | | | perform EtOAc |
| | | | | | | extraction but no buffer |
| | | | | | | wash; remove EtOAc, |
| | | | | | | add fresh EtOH and |
| | | | | | | repeat reaction |
| Ethanol/ | 2:1 | 18 | 20.10 | 0.04 | 63.67 | Double esterification; |
| Ethanol | | | | | | perform complete |
| | | | | | | workup after first rxn, |
| | | | | | | add fresh EtOH and |
| | | | | | | then 5 wt % H2SO4 and |
| | | | | | | repeat reaction |
| Propanol | 2:1 | 18 | 44.52 | 0.11 | 69.8 | |
| Propanol/ | 2:1 | 18 | 47.75 | 0.11 | 50.32 | |
| Propanol | | | | | | |
| Isopropanol | 2:1 | 18 | 38.58 | 0.08 | 129.94 | |
| Isopropanol/ | 2:1 | 18 | 57.20 | 0.01 | 90.22 | |
| Isopropanol | | | | | | |
| Butanol | 1:1 | 18 | 63.40 | 0.03 | 23.72 | |
| Butanol/ | 1:1 | 18 | 66.64 | 0.08 | 16.47 | |
| Butanol | | | | | | |
| Butanol | 1:1 | 2 | 54.90 | 0.26 | 51.91 | |
| Butanol | 1:1 | 3 | 50.86 | 0.01 | 34.46 | |
| Butanol | 1:1 | 6 | 57.60 | 0.03 | 32.27 | |
| Butanol | 1:1 | 24 | 63.67 | 0.06 | 29.9 | |
| Butanol | 0.5:1 | 18 | 56.25 | 0.07 | 44.38 | |
| Ethanol + Butanol | 2:0.5:1 | 18 | 42.90 | 0.11 | 71.16 | Mixed alcohol |
| | | | | | | esterification, ratio is |
| | | | | | | EtOH:BuOH:feedstock |
| Ethanol + Butanol | 1:0.5:1 | 18 | 48.02 | 0.03 | 64.36 | Mixed alcohol |
| | | | | | | esterification, ratio is |
| | | | | | | EtOH:BuOH:feedstock |
| Ethanol + Butanol | 1:0.5:1 | 24 | 44.52 | 0.06 | 63.12 | Mixed alcohol |
| | | | | | | esterification, ratio is |
| | | | | | | EtOH:BuOH:feedstock |
| Ethanol + Butanol | 1:0.5:1 | 24 | 48.02 | 0.04 | 52.16 | Mixed alcohol |
| | | | | | | esterification, ratio is |
| | | | | | | EtOH:BuOH:feedstock; |
| | | | | | | add BuOH after 6 h |

The data summarized in table 9 indicates that upon exposure to a composition according to a preferred embodiment of the present invention, the lignin-derived material from the LHDO concentrate treated at 55° C. for 18 hours yielded at least 14% of esterified lignin derivatives in every reaction. Alcohol chain length seems to be the primary factor in achieving higher yields, with methanol giving the lowest yields while butanol gave the highest. This is likely due to the improved stability of the ester product, as the carboxylic acid and ester exist in an equilibrium under reaction conditions. A comparison of n-propanol and isopropanol also indicates that formation of linear esters is favoured over branched. Furthermore, double esterification does not significantly impact recovered yields but led to consistent reduction in water content and/or TAN values, indicating that the hydrophobicity of the product has increased.

When used in this specification and claims, the terms “comprises” and “comprising” and variations thereof mean that the specified features, steps or integers are included. The terms are not to be interpreted to exclude the presence of other features, steps or components. The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

All publications, patents, patent applications and other documents cited in this application, including priority document CA3208558, are hereby incorporated by reference in their entireties for all purposes to the same extent as if each individual publication, patent, patent application or other document were individually indicated to be incorporated by reference for all purposes.

1. A method for converting a lignin-derived material into at least one esterified lignin derivative, said method comprising the steps of:
a providing said lignin-derived material, wherein the lignin-derived material is selected from lignin monomers; lignin depolymerization products; and combinations thereof;
b) providing an acidic composition having a pH of less than 1, said acidic composition comprising:
i) an acid selected from sulfuric acid, an alkylsulfonic acid, and an arylsulfonic acid; and
ii) an alcohol selected from a C₁-C₈ linear alcohol, a C₃-C₈ branched alcohol, and mixtures thereof;
c) combining said lignin-derived material with said acidic composition into a reaction mixture;
d) heating up said reaction mixture to a temperature ranging from 25° C. to 120° C.; and
e) allowing sufficient time of reaction to convert at least a portion of said lignin-derived material into said at least one esterified lignin derivative.

2. The method according to claim 1, wherein the alcohol is selected from methanol, ethanol, n-propanol, isopropanol, n-butanol, isobutanol, n-pentanol, neo-pentanol, isopentanol, isoamyl alcohol, and mixtures thereof.

3. The method according to claim 1, wherein the alcohol and the sulfuric acid are present in a molar ratio ranging from 2.85:1 (alcohol:sulfuric acid) to 10:1 (alcohol:sulfuric acid).

4. The method according to claim 1, wherein the alcohol and the sulfuric acid are present in a molar ratio ranging from 3:1 (alcohol:sulfuric acid) to 5:1 (alcohol:sulfuric acid).

5. The method according to claim 1, wherein the lignin-containing material results from a delignification reaction of a lignocellulosic material using a modified Caro's acid.

6. The method according to claim 1, wherein said at least one esterified lignin derivative is selected from alkyl malonate, alkyl maleate, alkyl succinate, alkyl oxalate, dialkyl malonate, dialkyl maleate, dialkyl succinate, dialkyl oxalate, alkyl vanillate, and alkylparaben.

7. The method according to claim 6, wherein said at least one esterified lignin derivative is selected from dibutyl malonate, dibutyl maleate, dibutyl succinate, and dibutyl paraben.

8. The method according to claim 1, wherein the alkylsulfonic acid is selected from methanesulfonic acid, ethanesulfonic acid, propanesulfonic acid, and combinations thereof.

9. The method according to claim 1, wherein the arylsulfonic acid is selected from toluenesulfonic acid, benzenesulfonic acid, and combinations thereof.

10. A method to convert lignin and hemicellulose depolymerized organics (LHDO) into at least one esterified lignin derivative and to recover such esterified lignin derivative, said method comprising the steps of:
a) providing a LHDO comprising a lignin-containing material resulting from a delignification reaction of a lignocellulosic material using a modified Caro's acid, wherein said lignin-containing material comprises a total acid content ranging from 35-70%, preferably, said lignin-containing material is present in an amount of at least 3%;
b) optionally, removing at least a portion of water present in said LHDO;
c) adding the LHDO to a flask containing a magnetic stir bar;
d) concentrating the material using a rotary evaporator until all volatile solvent is removed;
e) weighing the residue, and adding a required mass of alcohol solvent (preferably 1:1 or 8:1 alcohol:LHDO by weight);
f) placing the mixture in an oil bath on a heating stir plate, where the bath has a temperature set to either 25° C. or 100° C.;
g) stirring the reaction mixture at the desired temperature for up to 16 hours;
h) optionally, cooling the reaction mixture;
i) filtering the reaction mixture to remove precipitated solids;
j) rinsing the filtered precipitated solids with additional alcohol, and collecting, drying and weighing said solids;
k) concentrating the filtrate by evaporation and then transferring the resulting concentrated filtrate to a separatory funnel;
l) adding water and ethyl acetate, and extracting the product into an organic phase;
m) optionally repeating the extracting step;
n) collecting an organic phase;
o) extracting an aqueous phase;
p) optionally, combining the organic phases and transferring the combined organic phases back into the separatory funnel;
q) optionally, washing the combined organic phases with two portions of a pH 2 sulfate buffer solution;
r) optionally, drying the organic phase over MgSO₄, filtered into a round bottom flask, and evaporating the remaining volatiles; and
s) optionally, weighing the residue and calculating a yield.

11. The method according to claim 10, wherein the LHDO has an acid content ranging from 40-45% prior to the addition into the flask.

12. The method according to claim 1, wherein said lignin monomers are present in an amount ranging from 20 to 50 wt. % of said lignin-derived material.

13. The method according to claim 1, wherein said lignin depolymerization products are present in an amount ranging from 50 to 80 wt. % of said lignin-derived material.
